(19) United States
(12) Patent Application Publication
(10) Pub. No.: US 2018/0228399 A1
(43) Pub. Date: Aug. 16, 2018

(54) CAPNOGRAPHY WITH DECISION SUPPORT SYSTEM ARCHITECTURE

(71) Applicant: KONINKLIJKE PHILIPS N.V., EINDHOVEN (NL)

(72) Inventors: JOSEPH ALLEN ORR, PARK CITY, UT (US); LARA MARIE BREWER, BOUNTIFUL, UT (US); PAUL BRUCE GUNNESON, CHESHIRE, CT (US)

(73) Assignee: KONINKLIJKE PHILIPS N.V.

(21) Appl. No.: 15/751,354

(22) PCT Filed: Jul. 27, 2016
(86) PCT No.: PCT/IB2016/054469

§ 371 Date: Feb. 8, 2018

Related U.S. Application Data
(60) Provisional application No. 62/203,416, filed on Aug. 11, 2015.

(51) Int. Cl. A61B 5/097; A61M 16/00; G01N 33/497; G06F 17/18; A61B 5/00

(52) U.S. Cl. CPC A61B 5/097; A61M 16/021; G01N 33/497; A61M 2230/432; A61B 5/7235; A61B 5/7271; A61B 5/742; G06F 17/18

(57) ABSTRACT

A capnograph device (10) includes a carbon dioxide measurement component (20) and an electronic processor (30) programmed to generate a capnogram (40) comprising carbon dioxide level sample values measured as a function of time. End-tidal carbon dioxide (etC02) is determined from the capnogram, and an etC02 parameter quality index (etC02 PQI) (44) is computed using one or more quantitative capnogram waveform metrics computed from the capnogram. A respiration rate (RR) value is also determined from the capnogram, and a RR PQI (46) computed using the RR value and the etC02 PQI. A respiratory well-being index (RWI) (50) may be computed from the etC02 and RR values and the etC02 and RR PQI values. In some embodiments the one or more capnogram waveform metrics are computed from a capnogram histogram generated from the capnogram.

### FIELD

The following relates generally to the capnography arts, medical monitoring arts, and related arts.

### BACKGROUND

A capnography device monitors the concentration or partial pressure of carbon dioxide (CO₂) in respiratory gases. Capnography is commonly used in conjunction with mechanically ventilated patients in order to assess respiratory system status. A skilled anesthesiologist can evaluate the capnogram (that is, the CO₂ trend line as measured by a capnograph device) to assess respiratory health.

Capnography is increasingly used as a more generic vital sign for assessing patient health. For example, capnography may be used to monitor a patient who is breathing spontaneously and not undergoing mechanical ventilation, using a side stream capnograph device configuration in which respired air is sampled via a nasal cannula in conjunction with a dedicated sampling pump. In these broader contexts, medical personnel with limited expertise in anesthesiology are required to assess respiratory health on the basis of capnograph data. To facilitate this, it is common for the capnograph device to be programmed to output standard derived parameters, particularly respiration rate (RR) and end-tidal CO₂ (etCO₂). The RR is the breathing rate, quantified as the (quasi-)periodicity of the capnogram waveform. The etCO₂ is the partial pressure at the end of the exhalation phase. However, since the expired CO₂ is usually highest at the end of the exhalation phase, etCO₂ is commonly defined as the maximum observed CO₂ partial pressure over the breathing cycle.

While RR and etCO₂ are useful parameters, they do not capture the rich informational content of the capnogram waveform. To this end, it is also known to perform automated capnogram waveform analyses, designed to mimic clinical analyses that might be performed by a skilled anesthesiologist. For example, Colman et al., U.S. Pat. No. 8,412,655 and Colman et al., U.S. Pat. No. 8,414,488 disclose capnogram waveform analyses such as correlating pauses with apnea events, correlating a long downward slope of the capnogram waveform with possible partial airway obstruction, correlating a low capnogram waveform with possible low blood pressure, correlating a rounded capnogram waveform with a possible problem with the nasal cannula, or so forth. Based on such waveform analyses, the capnograph device may provide informational messages such as “open airway”, “check airway”, “check blood pressure”, “check cannula interface”, or so forth.

Capnogram waveform analyses provide richer information from the capnogram, but entail complex processing such as detecting the breath cycling, amplitude and period normalization, and segmenting regions of the capnogram waveform within each breath cycle. These complex analyses introduce numerous possible error mechanisms such as incorrect waveform segmentation or information loss during the normalization operations.

The following discloses a new and improved systems and methods that address the above referenced issues, and others.

### SUMMARY

In one disclosed aspect, a capnograph device comprises a carbon dioxide measurement component configured to measure respiratory carbon dioxide level, and an electronic processor programmed to: generate a capnogram signal comprising carbon dioxide level sample values measured by the carbon dioxide measurement component as a function of time; determine an end-tidal carbon dioxide (etCO₂) value from the capnogram signal; and compute an end-tidal carbon dioxide parameter quality index (etCO₂ PQI) value based on a waveform of the capnogram signal. A display component may be provided to display the etCO₂ value and the etCO₂ PQI value. In some embodiments the electronic processor computes the etCO₂ PQI by operations including generating a capnogram histogram by binning counts of carbon dioxide level sample values of the capnogram signal into carbon dioxide level bins.

In another disclosed aspect, a capnograph device comprises a carbon dioxide measurement component configured to measure respiratory carbon dioxide level, and an electronic processor programmed to: generate a capnogram signal comprising carbon dioxide level sample values measured by the carbon dioxide measurement component as a function of time; compute a quantitative capnogram waveform metric from the capnogram signal; determine an end-tidal carbon dioxide (etCO₂) value and a respiration rate (RR) value from the capnogram signal; compute an end-tidal carbon dioxide parameter quality index (etCO₂ PQI) value using the quantitative capnogram waveform metric; and compute a respiration rate parameter quality index (RR PQI) value using the RR value and the etCO₂ PQI.

One advantage resides in providing a capnograph device whose output more effectively assesses patient respiratory health.

Another advantage resides in providing a capnograph device outputting derived parameters characterizing the detailed capnogram waveform without requiring breath detection or segmentation of the capnogram waveform.

Another advantage resides in more accurate respiratory system status information from capnogram data.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 diagrammatically illustrates a capnograph device.

FIG. 2 diagrammatically illustrates an idealized capnogram and the corresponding capnogram histogram.

FIG. 3 diagrammatically plots the CO₂ fall time for an idealized capnogram waveform (top plot) and a capnogram with supplemental oxygen washout (bottom plot).

### DETAILED DESCRIPTION

In some embodiments disclosed herein, parameter quality indices are computed to quantitatively assess the reliability of the respiratory rate (RR) and end-tidal CO₂ (etCO₂) evaluated from the capnogram. A respiratory well-being index (RWI) may also be computed, based in part on the etCO₂ parameter quality index (etCO₂ PQI) and the RR parameter quality index (RR PQI). These parameter quality indices enable medical personnel to interpret the capnogram using conventional tools, especially the RR and etCO₂, but provide metrics (the quality control indices) to assist medical personnel in assessing whether the RR and etCO₂ are reliable data for making clinical decisions.

Further, in some embodiments the parameter quality indices are computed at least in part using a histogram of CO₂ value counts versus (binned) CO₂ level. This histogram is computed over a time interval encompassing several breaths. For example, the histogram is acquired over a 30 second time interval in one illustrative embodiment, which corresponds to about 6-10 breaths for a normal adult patient respiration interval of 3-5 seconds/breath (12-20 breaths per minute), up to 30 breaths for a rapidly respiring infant (respiration rate of 60 breaths per minute).

Advantageously, the capnogram histogram is computed without segmenting the waveform into different regions (e.g. inspiration, expiration) and without segmenting individual breath cycles (that is, without a breath detector). The capnogram histogram advantageously has a “standard” shape for a normally respiring patient, due to the typical capnogram pattern in which the CO₂ level is close to zero during the inspiration phase and close to its maximum (i.e. close to etCO₂ for the patient) during the expiration phase. These two phases define respective low and high regions of the disclosed capnogram histogram, with a third transitional histogram region in-between. Rich information about the capnogram waveform can be extracted from the capnogram histogram, without reliance upon the difficult and often imprecise task of segmenting the capnogram waveform into breath cycles which are then further segmented into inspiration and expiration time intervals. In particular, the etCO₂ PQI is computed primarily or entirely using the histogram. In some embodiments the etCO₂ PQI is computed further based on capnogram characteristics that can be quantified without segmenting the capnogram into inspiration and expiration regions. Illustrative embodiments of the RR PQI do rely upon breath detection and capnogram waveform segmentation, as the RR is intimately associated with (indeed defined by) the breath cycle. However, the RR PQI is optionally further based on the etCO₂ PQI thereby incorporating waveform information from the capnogram histogram.

The RWI is computed based on the etCO₂ and RR values, and further based on the etCO₂ PQI and RR PQI. Incorporating the PQI values into the RWI captures the recognition herein that a poor capnogram waveform is often an indication of poor respiratory health, rather than being an indicator of a capnograph measurement problem.

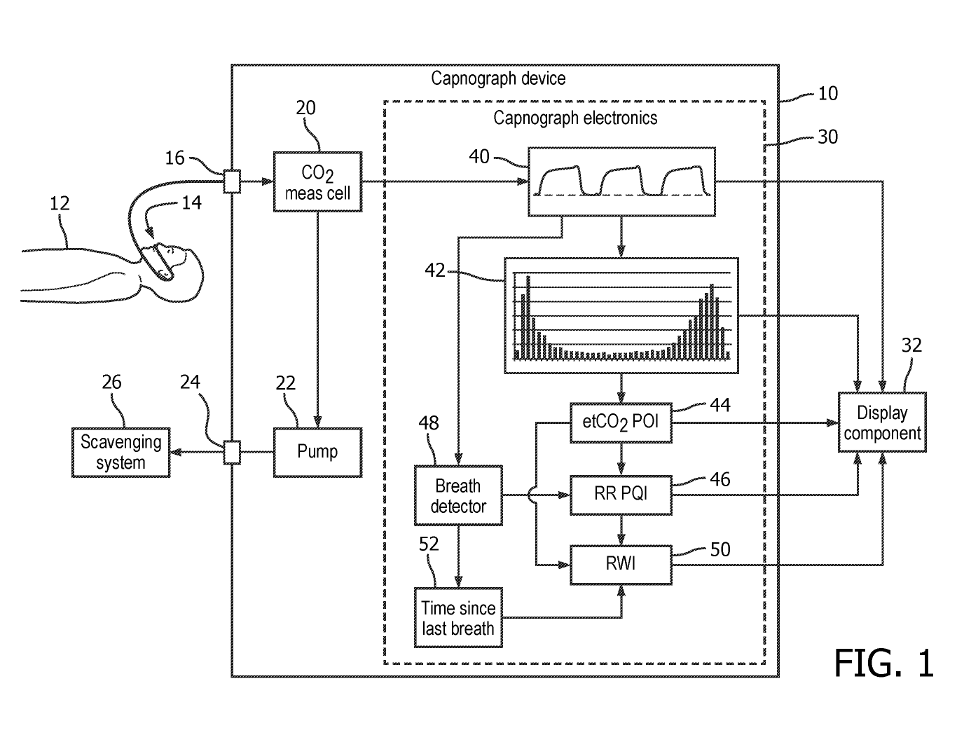

With reference to FIG. 1, an illustrative capnograph device 10 is connected with a patient 12 by a suitable patient accessory, such as a nasal cannula 14 in the illustrative example, or by an airway adaptor or so forth. The patient accessory 14 may optionally include one or more ancillary components, such as an air filter, water trap, or the like (not shown). In the illustrative capnograph 10, respired air is drawn from the patient accessory 14 into a capnograph air inlet 16 and through a carbon dioxide (CO₂) measurement component or cell 20 by an air pump 22. The air is then discharged via an air outlet 24 of the capnograph 10 to atmosphere or, as in the illustrative embodiment, is discharged through the air outlet 24 into a scavenging system 26 to remove an inhaled anesthetic or other inhaled medicinal agent before discharge into the atmosphere. The CO₂ measurement component or cell 20 may, for example comprise an infrared optical absorption cell in which carbon dioxide in the respired air drawn from the patient accessory 14 produces absorption that is detected by an infrared light source/detector assembly.

The illustrative capnograph device 10 has a sidestream configuration in which respired air is drawn into the capnograph device 10 using the pump 22, and the CO₂ measurement cell 20 is located inside the capnograph device 10. That is, the sidestream capnograph device 10 includes, as a unit, the carbon dioxide measurement component 20, the electronic processor 30, and the pump 22 connected to draw respired air though the carbon dioxide measurement component 20. The sidestream configuration is suitably used for a spontaneously breathing patient, i.e. a patient who is breathing on his or her own without assistance of a mechanical ventilator. In an alternative configuration, known as a mainstream configuration (not illustrated), the CO₂ measurement cell is located externally from the capnograph device housing, typically as a CO₂ measurement cell patient accessory that is inserted into the “mainstream” airway flow of the patient. Such a mainstream configuration may, for example, be employed in conjunction with a mechanically ventilated patient in which the CO₂ measurement cell patient accessory is designed to mate into an accessory receptacle of the ventilator unit, or is installed on an airway hose feeding into the ventilator. The disclosed approaches for quantitatively assessing parameter quality and patient respiratory well-being are readily applied either in conjunction with a sidestream capnograph device (as in the illustrative example of FIG. 1) or in conjunction with a mainstream capnograph device.

With continuing reference to FIG. 1, the capnograph device 10 (in either the illustrative sidestream configuration or in the alternative mainstream configuration) includes capnograph electronics 30 which provide power and control for operating the CO₂ measurement cell 20 and (in the sidestream configuration) the pump 22. Note that the power and control links are not illustrated in diagrammatic FIG. 1. The capnograph electronics 30 additionally perform processing of the CO₂ signal output by the CO₂ measurement cell 20, as diagrammatically indicated in FIG. 1 and as described herein. Clinical data output by the capnograph 10 are displayed on a display component 32, stored in an electronic medical record (EMR) or the like, or otherwise utilized. The display component 32 may be a component of the capnograph or, as illustrated in FIG. 1, the display component 32 may be an external display component connected to the capnograph 10. For example, the external display component 32 may be a multi-function bedside patient monitor and/or a nurses' station patient monitor or so forth. It will be further appreciated that the capnograph may include numerous other components not illustrated in simplified diagrammatic FIG. 1, such as a pressure gauge, flow meter, and so forth.

The capnograph electronics 30 may be variously implemented, such as by a suitably programmed electronic processor, e.g. a microprocessor or microcontroller of the capnograph 10. While a single electronics unit 30 is illustrated, it is alternatively contemplated to employ various combinations of electronics, for example different electronic components may be operatively interconnected to implement a pump, power supply, infrared light source and detector, power supply (for the CO₂ measurement cell 20), analog-to-digital conversion circuitry (to sample the infrared light detector of the CO₂ measurement cell 20), and so forth. Still further, it is contemplated for the electronics that perform the capnograph data processing to be disposed outside of the capnograph device itself. For example, the capnograph data processing may be performed by electronics in another device (for example, the computer of a nurses' station that receives the CO₂ signal from the measurement cell 20, or that receives a capnogram generated by the capnograph device and performs further processing). It will be still further appreciated that the capnograph data processing disclosed herein as being performed by the capnograph electronics 30 may be embodied by a non-transitory storage medium storing instructions that are readable and executable by the microprocessor, microcontroller, or other electronic processor to perform the disclosed capnograph data processing. Such non-transitory storage media may, by way of non-limiting illustration, include a hard disk drive or other magnetic storage medium, a flash memory, read-only memory (ROM) or other electronic storage medium, an optical disk or other optical storage medium, various combinations thereof, or so forth.

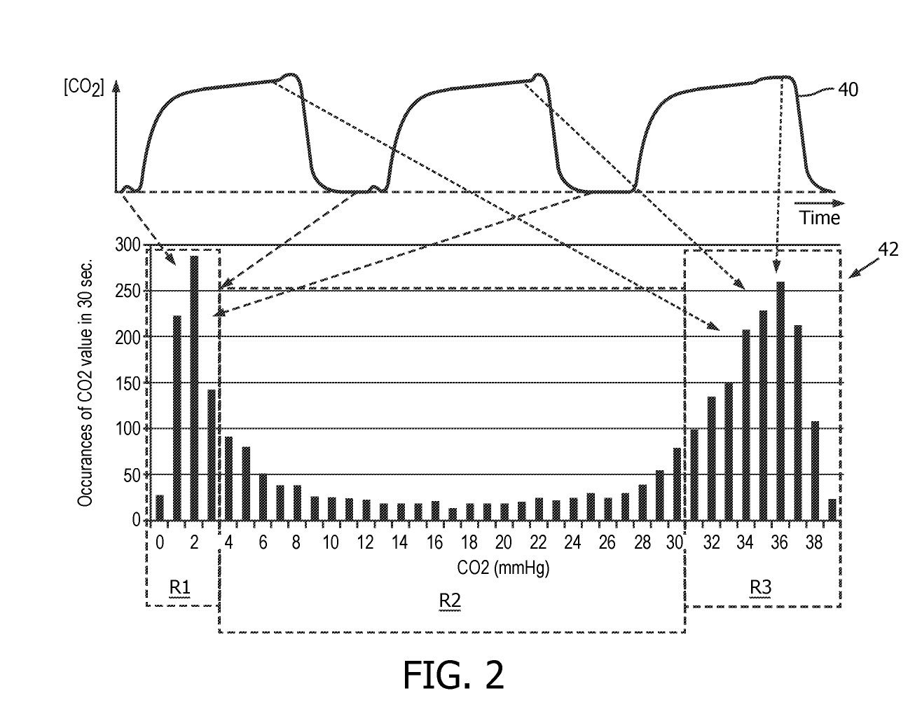

With continuing reference to FIG. 1 and with further reference to FIG. 2, an illustrative embodiment of the capnograph data processing performed by the capnograph electronics 30 (or alternatively in whole or in part by a nurses' station monitor, bedside patient monitor, or other device with a suitably programmed electronic data processor) is diagrammatically shown in FIG. 1. The CO₂ signal is sampled and optionally corrected for factors such as the presence of interfering gases (e.g. nitrous oxide), barometric pressure, and so forth in order to generate a capnogram 40. The capnogram 40 is a signal representing the partial pressure or concentration of carbon dioxide, denoted in FIG. 2 as [CO₂], as a function of time. Diagrammatic FIG. 2 illustrates the capnogram 40 as an idealized waveform for a healthy patient, in which every breath is identical and exhibits near-zero [CO₂] during the inspiratory phase and a well-defined maximum [CO₂] that rises gradually over the expiratory phase and terminates in a maximum [CO₂] corresponding to end-tidal CO₂, and in which the etCO₂ is the same for every breath. In practice, it will be understood that the capnogram 40 for a real patient usually deviates significantly from this idealized curve due to numerous factors such as non-uniform breathing, talking, coughing, possible chronic lung problems in the case of an ill patient, or so forth. In the capnogram of a real patient, the etCO₂ may vary from breath to breath. The illustrative idealized example of FIG. 2 further assumes a constant respiration rate. Again, in a real patient, the RR is generally not constant the RR can increase significantly due to excitement or exertion, may slow during rest periods, may stop entirely during a sleep apnea episode, and/or may generally vary significantly due to various respiratory ailments or other medical conditions.

With continuing reference to FIGS. 1 and 2, the capnograph electronics 30 are programmed to compute a capnogram histogram 42 from the capnogram 40. The capnogram histogram 42 is a histogram of CO₂ sample values (y-axis) versus CO₂ level (x-axis). The capnogram histogram 42 is computed for a sliding window of duration 30 seconds (for illustrative FIG. 2; other window sizes are contemplated, preferably of a duration long enough to encompass several breaths). By way of illustrative example, if the CO₂ measurement cell 20 acquires samples at 10 msec intervals (100 samples per second) and the window is 30 seconds then, for each capnogram sample in the 30 second window (consisting of 3000 points) the bin corresponding to the CO₂ value for the point is incremented. In the capnogram histogram of a typical capnogram, there is a lower baseline region during inspiration and an elevated CO₂ region during expiration. Between these two regions, there are a set of points that make up the rising and falling edges of the capnogram. More particularly, as delineated in FIG. 2, three regions R1, R2, R3 can be defined. Region R1 of the histogram 42 includes the points in the capnogram 40 measured by the CO₂ measurement cell 20 during the inspiratory phase of the breath. In the illustrative example of FIG. 2, Region R1 includes the bins from 0 to 3 mmHg. Region R2 of the histogram 42 includes all the points from the capnogram 40 forming the rising and falling edges in the capnogram 40. In the illustrative example of FIG. 2, Region R2 includes the bins from 4 to 30 mmHg. Finally, Region R3 of the histogram 42 includes the points in the capnogram 40 measured during the expiratory phase of the breath. In the illustrative example of FIG. 2, Region R3 includes all the bins from 31 to 39 mmHg.

The capnogram histogram of a typical capnogram has certain characteristics. The histogram for a typical capnogram will have a higher number of occurrences of CO₂ sample values in the bins of Region R1 and Region R3, and the number of occurrences in the bins of Region R2 should be lower than the number of occurrences in Regions R1 and R3. That is, the capnogram histogram 42 has a peak in lower Region R1 and a peak in upper Region R3, and a valley in the intermediate Region R2. Further, the peak in upper Region R3 is typically more spread-out than the peak in Region R1, as seen in the idealized capnogram histogram 42 of FIG. 2. The spread in the peak in upper Region R3 is caused by the slope of the capnogram 40 during the exhalation phase, with the highest CO₂ value typically occurring at the end of the breath (i.e. end-tidal point). This slope of the capnogram waveform 40 is reflected in spreading of the points making up the peak in upper Region R3 of the capnogram histogram 42. Such spreading may additionally or alternatively be caused by the usual situation in which every breath does not have the same peak CO₂ value (or, said another way, etCO₂ varies from breath to breath). The difference in etCO₂ value for each breath is reflected in spreading of the peak in upper Region R3. By contrast, during the inspiration phase of the capnogram the CO₂ level usually falls to a flat baseline level that is close to zero, and exhibits little variation from breath to breath, leading to a narrower peak in the lower Region R1 of the histogram 42.

The capnogram histogram 42 is computed from the capnogram 40 in the sliding window, with a new histogram computed every few seconds, e.g. every 5 seconds in one illustrative example employing a 30 second window. There is no attempt to synchronize the window with an integer number of breaths, but the window is preferably large enough to encompass several breaths (e.g. for a normal adult patient respiration interval of 3-5 seconds/breath the illustrative 30 sec window encompasses 6-10 breaths). By re-computing the histogram on a shorter time interval than the window size (e.g. every 5 sec using a 30 sec window) the successive histogram windows significantly overlap providing for a smoothing effect as a function of time. Since there is no synchronization with the breath cycling, there is no need to employ a breath detector in constructing the capnogram histogram 42, and the determination of the histogram 42 is a very fast CO₂ sample binning process.

An end-tidal carbon dioxide (etCO₂) value and a respiratory rate (RR) value are determined from the capnogram signal 40. Substantially any technique to detect a signal maximum can be used to detect the etCO₂ value. For example, in some embodiments, the etCO₂ value is determined from the capnogram signal 40 by analysis of the histogram 42 derived from the capnogram signal 40. In this approach, the CO₂ level of the highest CO₂ level bin having a non-zero sample count provides an etCO₂ value. Similarly, substantially any technique to determine periodicity of a signal can be used to detect the RR value. For example, the RR value can be determined by detecting breaths using the breath detector 48 and thereby determining breath intervals (the RR being the inverse of the average breath interval). Alternatively, a Fast Fourier Transform (FFT) can be applied to determine the RR value in the frequency domain.

With continuing reference to FIG. 1, the capnogram histogram 42 is used to compute an end-tidal CO₂ parameter quality index (etCO₂ PQI) 44. This index is computed as a weighted sum of parameters derived from the capnogram histogram 42, and optionally also from the capnogram 40 itself. The parameters included in the weighted sum are suitably chosen as relevant criteria in determining the confidence in the etCO₂ measurement obtained from the capnogram 40. In one illustrative embodiment, the etCO₂ PQI 44 is computed from parameters including (1) a metric of the portion of the histogram 42 that is above the baseline; (2) a metric of the difference between the maximum CO₂ in Region R3 and the CO₂ level in Region R3 having the highest histogram counts; (3) a metric comparing the Region R3 count versus the Region R2 count; (4) a metric of the fraction of the total counts in Region R3; and (5) a metric of the CO₂ fall time.

The metric of the portion of the histogram 42 that is above the baseline characterizes the portion of the histogram that is in Region R3 as compared with Region R1. This metric is large for a normal capnogram, but may be low in the case of a poor capnogram waveform having an inconsistent expiratory plateau.

The metric of the difference between the maximum CO₂ in Region R3 and the CO₂ level in Region R3 having the highest histogram counts is expected to be small because the end-tidal point should have the largest CO₂ value and a CO₂ level bin at or close to etCO₂ should also have a large number of counts since the expiratory plateau usually flattens as it approaches the end-tidal point. This metric may be computed from the difference between the CO₂ level of the bin of Region R3 having a non-zero count and the CO₂ level of the bin of Region R3 storing the highest count.

The metric comparing the upper Region R3 count versus the intermediate Region R2 count quantifies the expectation that a sharp transition should be present from the inspiratory phase to the expiratory phase in the capnogram 40. In such a case, the intermediate Region R2 count is low and the upper Region R3 count is high. However, since there are more bins in intermediate Region R2 than in upper Region R3, this metric may preferably be quantified using the average count over all bins of Region R2, and likewise using the average count over all bins of Region R3.

The metric of the fraction of the total counts in upper Region R3 should be high since a large portion of the capnogram waveform consists of the expiratory phase. This metric may be computed using the ratio of the total counts in upper Region R3 to the total counts in the capnogram histogram 42.

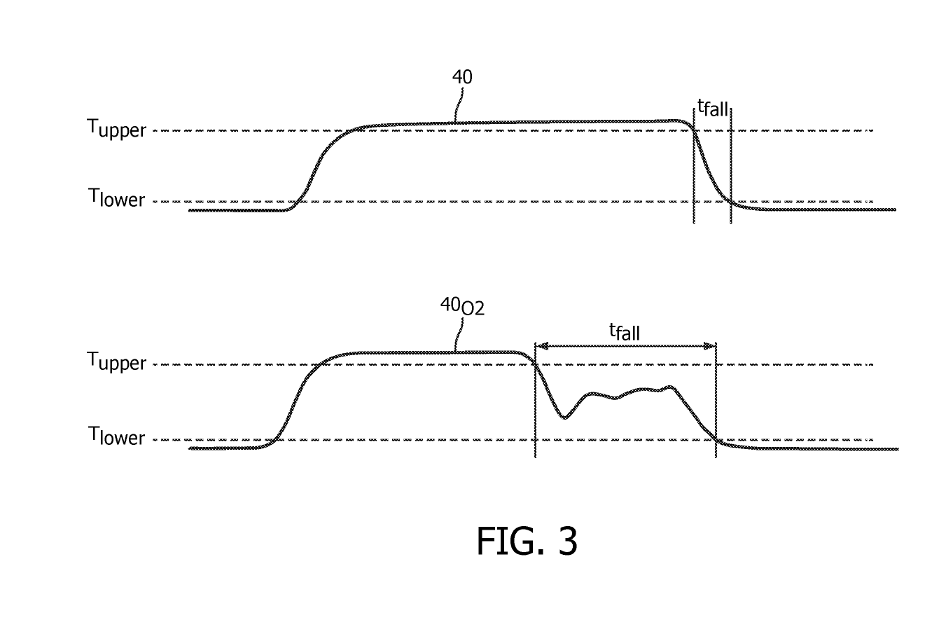

With brief reference to FIG. 3, the metric of the CO₂ fall time differs from the previous four metrics contributing to the illustrative etCO₂ PQI in that the metric of the CO₂ fall time is computed from the capnogram 40 rather than from the capnogram histogram 42. The metric of the CO₂ fall time is useful for detecting when the capnogram waveform is washed out due to the effect of supplemental oxygen. This is illustrated in FIG. 3. The top plot of FIG. 3 shows the expiratory plateau for the same idealized capnogram 40 as is shown in FIG. 2. The CO₂ fall time is computed as the time interval from when a high CO₂ level falls below an upper threshold Tᵤₚₚₑᵣ until the CO₂ level decreases below a lower threshold T_{lower}. This CO₂ fall time is indicated as t_{fall} in the top plot of FIG. 3 showing the idealized capnogram 40. It is seen that t_{fall} is relatively short. By contrast, FIG. 3 bottom plot shows a capnogram 40_{O2} which exhibits supplemental oxygen washout. In this case, the transition from Tᵤₚₚₑᵣ to T_{lower} is much longer.

It will be noted that the CO₂ fall time can be determined without performing breath detection, and without segmenting the capnogram waveform into inspiratory and expiratory phases. For example, in the illustrative example the CO₂ fall time is computed by identifying when a high CO₂ level falls below Tᵤₚₚₑᵣ and then when it falls below T_{lower}.

With returning reference to FIG. 1, the etCO₂ PQI 44 is suitably computed as a weighted sum of these metrics (and/or other metric that correlate with the reliability of the etCO₂ measurement by capnography). That is:

$${et CO}_{2} PQI = {\sum }_{i} {W}_{i} * {S}_{i}$$

where the index i ranges over the metrics contributing to etCO₂ PQI 44, Sᵢ is the score (i.e. value) of the ith metric, and Wᵢ is a weight for the ith metric. The weights may be generated manually (e.g. based on assessment by a skilled pulmonologist of the relative importance of the various metrics) or by performing machine learning using a training set of representative capnograms each labeled by a skilled pulmonologist as to reliability of the etCO₂ value obtained from the training capnogram.

The five metrics contributing etCO₂ PQI in the example are merely illustrative. More generally, it will be appreciated that the capnogram histogram 42 is expected to exhibit a large narrow peak in a lower Region R1 corresponding to the inspiration phase of the respiratory cycle, a large slightly broader peak in an upper Region R3 corresponding to the expiratory phase, and a deep valley in an intermediate Region R2 corresponding to transitions from inspiration-to-expiration and from expiration-to-inspiration. Deviations from this basic histogram shape are expected when the capnogram waveform is degraded, and consequently etCO₂ values are expected to be less reliable. Various metrics can be constructed and optimized using histograms constructed for training capnograms in order to quantitatively characterize metrics to assess the histogram shape and hence the capnogram waveform. The optimal choice of metrics, and their weights, depends on the capnograph device and its connection to the patient, the demographic being monitored, the desired sensitivity (e.g. how “bad” should the capnogram waveform be before the etCO₂ PQI starts to significantly decrease), and so forth. In some embodiments the metrics may be optimized for different patient connections (e.g. nasal cannula versus airway adaptor), different patient breathing conditions (e.g. spontaneous breathing versus various mechanical ventilation modes), or so forth. The capnogram histogram shape reflects the capnogram waveform, so quantitative metrics of the histogram provide assessment of the capnogram waveform quality without the need to detect breath intervals in the capnogram and without the need to segment the capnogram into inspiration and expiration phases. In the illustrative example, one metric (CO₂ fall time) is extracted directly from the capnogram 40 rather than from the capnogram histogram 42, but this is still done without performing breath detection or segmenting the capnogram into breathing phases. The computations are fast, and can be performed in real-time (that is, with a delay of a few tens of seconds, a few seconds, or less).

With continuing reference to FIG. 1, a respiratory rate parameter quality index (RR PQI) 46 is also determined. The RR, and the RR PQI, both depend on detecting breaths, and hence receive as input the breath intervals detected in the capnogram 40 by a breath detector 48. The RR PQI 46 is suitably determined as a weighted sum of metrics including, by way of illustrative example: respiratory rate (RR), a metric of expiration time/inspiration time ratio (IE ratio), a metric quantifying invalid peak counts in the capnogram of a breath, a capnogram carbon dioxide level dynamic range metric, and a metric of how close the inspiratory CO₂ level is to zero. The RR and IE ratio values should be in reasonable ranges (e.g. RR around 12-20 breaths per minute for an adult) so values falling significantly outside the reasonable reduce the RR PQI 46. Extra (invalid) peaks can result in erroneous breath detections, hence more invalid peaks reduces the RR PQI. The capnogram dynamic range (maximum CO₂ level minus minimum CO₂ level) impacts the signal strength, so a low dynamic range reduces RR PQI. Similarly, the CO₂ level should be close to zero during inspiration; whereas, a higher CO₂ level during inspiration makes breath detection more difficult leading to a lower value for the RR PQI 46.

In the illustrative embodiment of FIG. 1, the RR PQI 46 is also determined based on the etCO₂ PQI 44 which serves as an additional metric in the weighted sum. The etCO₂ PQI 44 is a metric of “normalcy” of the capnogram waveform. Since a highly abnormal capnogram waveform makes breath detection more difficult, a lower value for the etCO₂ PQI 44 results in a lower RR PQI value as well. Employing the etCO₂ PQI 44 as an input metric to the RR PQI 46 advantageously re-uses the etCO₂ PQI 44 in assessing reliability of the RR.

The RR PQI 46 is again suitably computed as a weighted sum of the contributing metrics:

$$RRPQI = {\sum }_{i} {W}_{i} * {S}_{i}$$

where the index i ranges over the metrics contributing to RR PQI 46, Sᵢ is the score (i.e. value) of the ith metric, and W_{W} is a weight for the ith metric. The weights again may be generated manually or by performing machine learning using a training set of representative capnograms labeled as to RR reliability. The metrics contributing RR PQI in the example are again merely illustrative, and additional or other metrics are contemplated.

In some embodiments, a respiratory well-being index (RWI) 50 is also computed, which represents a quality score to assess the respiratory well-being of a patient using the capnogram 40. The RWI 50 is designed to help medical personnel evaluate the overall respiratory well-being of the patient. RWI 50 may also be used to identify non-intubated patients who are at risk for hypoventilation due to central or obstructive apnea, such as during procedural sedation. In a suitable embodiment, metrics that serve as weighted inputs to the RWI 50 include the measured RR and etCO₂ and the corresponding RR PQI 44 and etCO₂ PQI 46. In general, if either the RR or etCO₂ are outside their respective normal ranges then this lowers the RWI 50. A lower RR PQI 44 or lower etCO₂ PQI 46 also lowers the RWI 50. In some embodiments, a time-since-last-breath metric is also incorporated into the RWI 50 in order to facilitate its use in detecting airway obstruction or apnea episodes. For example, the time-since-last-breath may be quantified from the capnogram 40 by a block 52 assessing the time since last elevated CO₂ level.

The indices 44, 46, 50 are suitably re-calculated each time the capnogram histogram 42 is updated, e.g. every 5 seconds in the illustrative example. Since the illustrative histogram calculation window is 30 seconds, the first calculation of the indices 44, 46, 50 is performed after 30 seconds of the capnogram 40 are acquired.

If the capnograph device 10 is programmed to provide informational messages based on the RR and etCO₂ values, then the indices 44, 46, 50 may optionally be used to suppress these informational messages when the underlying RR or etCO₂ is unreliable as indicated by the corresponding PQI. By way of non-limiting illustration, in one contemplated embodiment the messaging scheme of Table 1 is employed, with the outputs only being displayed when RWI is lower than some threshold value.

TABLE 1

| | Message when the | Message when the |
|---|---|---|
| | parameter is above the | parameter is below the |
| Parameter | “Normal” range | “Normal” range |
| etCO₂ | “Hypo-ventilation” | “Shallow breaths” “try |
| | | chin lift” |
| etCO₂ PQI Score | N/A | “Shallow breaths” “try |
| | | chin lift” |
| RR | “Patient anxious” | “Low resp rate”, |
| | (do not show message if RR | “prompt patient to |
| | PQI is low) | breathe” |
| RR PQI score | N/A | “Unstable breathing” |
| Time since high | “Long time since breath”, | N/A |
| CO₂ signal | “prompt patient to breathe” | |

In this illustrative messaging scheme, the “patient anxious” message is suppressed if the RR PQI 46 is below a threshold value.

In addition to (or in place of) computing and displaying (on the display component 32) values of probative parameters such as etCO₂, RR, etCO₂ PQI 44, RR PQI 46, and/or RWI 50, it is contemplated to display on the display component 32 the capnogram histogram 42 itself. As previously discussed, the capnogram histogram 42 embodies substantial information about the capnogram waveform in a format that may be more readily perceived by medical personnel as compared with reading a display of the capnogram 40 (which may optionally also be displayed on the display 32, e.g. as a trend line). One advantage of displaying the capnogram histogram 42 as compared with displaying a trend line of the capnogram 40 is that the trend line is typically scrolled horizontally, whereas the capnogram histogram 42 does not scroll and is updated, e.g. every 5 seconds with substantial overlap between successive updates due to the large window overlap between successive updates (e.g. with a 30 second window and 5 sec updates, each successive histogram is derived from 25 seconds of the same capnogram data that was used to generate the immediately previous histogram and only 5 seconds of new capnogram data).

The invention has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof.

1. A capnograph device comprising:
a carbon dioxide measurement component configured to measure respiratory carbon dioxide level; and
an electronic processor programmed to:
generate a capnogram signal comprising carbon dioxide level sample values measured by the carbon dioxide measurement component as a function of time;
determine an end-tidal carbon dioxide (etCO₂) value from the capnogram signal; and
compute an end-tidal carbon dioxide parameter quality index (etCO₂ PQI) value based on a waveform of the capnogram signal, wherein the electronic processor computes the etCO₂ PQI using at least one of:
a metric of the CO₂ fall time from an upper carbon dioxide level threshold (Tᵤₚₚₑᵣ) to a lower carbon dioxide level threshold (T_{lower}), and
by operations including generating a capnogram histogram by binning counts of carbon dioxide level sample values of the capnogram signal into carbon dioxide level bins; and
a display component configured to display the etCO₂ value and the etCO₂ PQI value.

2. The capnograph device of claim 1 wherein the electronic processor computes the etCO₂ PQI using a metric of the CO₂ fall time from an upper carbon dioxide level threshold (Tᵤₚₚₑᵣ) to a lower carbon dioxide level threshold (T_{lower}).

3. The capnograph device of claim 1 wherein the electronic processor is further programmed to:
determine a respiration rate (RR) value from the capnogram signal; and
compute a respiration rate parameter quality index (RR PQI) value using the RR value and the etCO₂ PQI.

4. The capnograph device of claim 3 wherein the electronic processor computes the RR PQI further using at least one of (i) a metric of expiration time/inspiration time ratio, (ii) a metric quantifying invalid peak counts in the capnogram of a breath, and (iii) a metric quantifying capnogram carbon dioxide level dynamic range.

5. The capnograph device of claim 1 wherein the electronic processor is further programmed to:
determine a respiratory well-being index (RWI) using the RR value, the etCO₂ value, the etCO₂ PQI, and the RR PQI.

6. The capnograph device of claim 5 wherein the electronic processor computes the RWI further using a time since last elevated CO₂ level determined from the capnogram.

7. The capnograph device of claim 1 wherein the electronic processor computes the etCO₂ PQI by operations including generating a capnogram histogram by binning counts of carbon dioxide level sample values of the capnogram signal into carbon dioxide level bins.

8. The capnograph device of claim 1 further comprising:
a display component configured to display at least the determined etCO₂ value and the computed etCO₂ PQI value.

9. A capnograph device comprising:
a carbon dioxide measurement component configured to measure respiratory carbon dioxide level; and
an electronic processor programmed to:
generate a capnogram signal comprising carbon dioxide level sample values measured by the carbon dioxide measurement component as a function of time;
compute a quantitative capnogram waveform metric from the capnogram signal;
determine an end-tidal carbon dioxide (etCO₂) value and a respiration rate (RR) value from the capnogram signal;
compute an end-tidal carbon dioxide parameter quality index (etCO₂ PQI) value using the quantitative capnogram waveform metric; and
compute a respiration rate parameter quality index (RR PQI) value using the RR value and the etCO₂ PQI.

10. The capnograph device of claim 9 wherein the electronic processor is programmed to compute the quantitative capnogram waveform metric from the capnogram signal by operations including:
generating a capnogram histogram from the capnogram signal wherein the capnogram histogram comprises carbon dioxide level bins storing corresponding counts of carbon dioxide level sample values in the capnogram signal; and
computing the quantitative capnogram waveform metric from the capnogram histogram.

11. The capnograph device of claim 10 wherein the electronic processor is programmed to determine the etCO₂ value from the capnogram signal by operations including:
determining the etCO₂ value from the capnogram histogram as the carbon dioxide level of the highest carbon dioxide level bin storing a non-zero count of carbon dioxide level sample values.

12. The capnograph device of claim 10, wherein the quantitative capnogram waveform metric includes a metric of the portion of the capnogram histogram that is above a baseline.

13. The capnograph device of claim 10 wherein the quantitative capnogram waveform metric includes a metric of the difference between the CO₂ level of a highest carbon dioxide level bin in an upper region of the capnogram histogram storing a non-zero count and the CO₂ level of the carbon dioxide level bin in the upper region of the capnogram histogram storing the highest count of carbon dioxide level sample values.

14. The capnograph device of claim 10 wherein the quantitative capnogram waveform metric includes a metric comparing counts stored in all carbon dioxide level bins of an upper region of the capnogram histogram and counts stored in all carbon dioxide level bins of an intermediate region of the capnogram histogram.

15. The capnograph device of claim 10 wherein the quantitative capnogram waveform metric includes a metric of the portion of all counts of the capnogram histogram in an upper region of the capnogram histogram.

16. The capnograph device of claim 9 wherein the electronic processor is programmed to compute the etCO₂ PQI using a metric of the CO₂ fall time from an upper carbon dioxide level threshold (Tᵤₚₚₑᵣ) to a lower carbon dioxide level threshold (T_{lower}).

17. The capnograph device of claim 9 wherein the electronic processor computes the RR PQI further using at least one of (i) a metric of expiration time/inspiration time ratio, (ii) a metric quantifying invalid peak counts in the capnogram of a breath, and (iii) a metric quantifying capnogram carbon dioxide level dynamic range.

18. The capnograph device of claim 9 wherein the electronic processor is further programmed to:
determine a respiratory well-being index (RWI) using the RR value, the etCO₂ value, the etCO₂ PQI, and the RR PQI.

19. The capnograph device of claim 18 wherein the electronic processor computes the RWI further using a time since last elevated CO₂ level determined from the capnogram.

20. The capnograph device of claim 9 further comprising:
a display component configured to display at least one of the determined etCO₂ value, the determined RR value, the computed etCO₂ PQI, and the computed RR PQI value.
